(19) United States
(12) Patent Application Publication
(10) Pub. No.: US 2005/0080471 A1
(43) Pub. Date: Apr. 14, 2005

(54) Lead body construction

(76) Inventors: Yougandh Chitre, Valencia, CA (US); Gene A. Bornzin, Simi Valley, CA (US); Sheldon Williams, Green Valley, CA (US)

Correspondence Address: PACESETTER, INC., 15900 VALLEY VIEW COURT, SYLMAR, CA, 91392-9221, US

(21) Appl. No.: 10/652,996

(22) Filed: Aug. 28, 2003

(51) Int. Cl. A61N 1/05

(52) U.S. Cl. 607/122

(57) ABSTRACT

An implantable cardiac stimulation lead system for use with a stimulation device includes a lead body preferably having an outer diameter of less than 4 French and formed of a biocompatible material having a Young's modulus of at least 20,000 pounds per square inch. In the lead body, an inner insulator is composed of a suitable polymer such as PTFE or ETFE with an outer peripheral surface formed with a helical groove containing a conductor extending between a proximal connector and a distal tip electrode, and an outer insulator composed of polyurethane or silicone rubber or a combination thereof is positioned around the inner insulator. The lead system may be formed with a lumen for receiving a guidewire longitudinally through the lead body. A pair of insulated conductors may be received in the helical groove, one extending to the distal tip electrode, the other extending to a ring electrode proximally spaced from the distal tip electrode.

### FIELD OF THE INVENTION

The present invention relates generally to implantable medical devices for providing stimulating pulses to selected body tissue, and more particularly, to the construction of lead assemblies in which the lead body diameter is minimized. Further, the present invention relates generally to implantable cardiac leads, and more particularly to endocardial leads having relatively small diameter lead bodies.

### BACKGROUND OF THE INVENTION

Over the past few years, there has been a substantial effort to reduce the diameter of endocardial pacing, cardioversion and defibrillation leads. Reduction in lead body diameter facilitates placement of multiple leads through a single blood vessel and also minimizes interference between the lead body and the tricuspid valve, in the case of leads implanted in the right ventricle. In addition, a reduction in lead body size has generally been accompanied by a reduction in the lead body's flexural stiffness or bending moment. This reduction in lead body flexural stiffness has generally been seen as an advantage, in that it reduces the possibility of perforation of heart tissue by the distal tip of the lead.

Although it will become evident to those skilled in the art that the present invention is applicable to a variety of implantable medical devices utilizing pulse generators to stimulate selected body tissue, the invention and its background will be described principally in the context of a specific example of such devices, namely, cardiac pacemakers for providing precisely controlled stimulation pulses to the heart. However, the appended claims are not intended to be limited to any specific example or embodiment described herein.

Pacemaker leads form the electrical connection between the cardiac pacemaker pulse generator and the heart tissue which is to be stimulated. As is well known, the leads connecting such pacemakers with the heart may be used for pacing, or for sensing electrical signals produced by the heart, or for both pacing and sensing in which case a single lead serves as a bidirectional pulse transmission link between the pacemaker and the heart. An endocardial type lead, that is, a lead which is inserted into a vein and guided therethrough into a cavity of the heart, includes at its distal end an electrode designed to contact the endocardium, the tissue lining the inside of the heart. The lead further includes a proximal end having a connector pin adapted to be received by a mating socket in the pacemaker. A flexible, coiled conductor surrounded by an insulating tube or sheath typically couples the connector pin at the proximal end and the electrode at the distal end.

The implantable cardiac stimulation leads with which the present invention is concerned may take the form of pacemakers capable of pacing and sensing in at least one chamber of the heart. Indeed, the present invention, may relate to a programmable dual chamber pacemaker wherein the basic configuration of the pacemaker, e.g. unipolar or bipolar, can be changed, including the grounding configuration and ground potentials used within the pacemaker.

Generally, a heart stimulator, commonly known as a “pacemaker” or “pacer”, uses one or two flexible leads having one end connected to the pacer and the other end connected to electrodes placed in close proximity to the heart. These leads are used to stimulate or pace the heart. Also, these leads are used to sense the heart activity by picking up electrical signals from the heart.

In order to properly pace or sense, the pacer has to be able to deliver a stimulating pulse to the heart or sense an electrical signal from the heart, and this requires that there be an electrical return path. If, within a given heart chamber, a unipolar lead is used—containing a single conductor—the return path is the conductive body tissue and fluids. The return path is connected to the pacer by connecting the pacer electrical common or ground to the pacer metal enclosure, typically referred to as the pacer case or housing. The case, in turn, makes contact with the body tissue and/or fluids.

An alternative solution to using a unipolar lead in a given heart chamber is to use a double lead/electrode in the heart chamber, known as a bipolar lead. In a bipolar lead, a second conductor is spiraled over and insulated from a first conductor along the length of the lead. At the distal end of the lead, one of the conductors is connected to a first electrode, referred to as the “tip” electrode, and the second conductor is connected to a second electrode, referred to as a “ring” electrode. The ring electrode is generally situated about 10 to 20 mm from the tip electrode. The tip electrode is typically placed in contact with heart tissue, while the ring electrode is in electrical contact with the blood. Because both body tissue and fluids are conductive, the ring electrode of a bipolar lead, in contact with the body fluids, serves as an electrical return for both pacing and sensing.

As indicated, pacing or sensing using the pacer case or enclosure as part of the electrical return path is known as unipolar pacing or sensing. Pacing or sensing using the lead ring electrode and associated lead conductor as the electrical return path is known as bipolar pacing or sensing. There are numerous factors to consider when deciding whether unipolar or bipolar pacing and/or sensing should be used. Bipolar pacing has, in general, the advantage of requiring less energy than unipolar pacing. Further, bipolar sensing is less prone to crosstalk and myopotential sensing than is unipolar sensing. Crosstalk generally refers to a pacer mistakenly sensing a heart activity in one heart chamber immediately after the other chamber is paced. Bipolar sensing reduces crosstalk resulting from a pacing stimulus in the opposite chamber. Bipolar pacing is preferred if pectoral or diaphragmatic stimulation occurs.

Unipolar pacing and sensing offers the advantage, in general, of simpler circuitry within the pacemaker and a smaller diameter lead. Some physicians prefer unipolar over bipolar pacing and/or sensing as a function of other implantation and heart conditions. Depending on the lead orientation, unipolar sensing may be preferable to bipolar sensing. In any event, cardiac pacing leads, intended to be placed in the chambers of the heart or the coronary venous system are subjected to a series of tortuous bends. The leads must have the flexibility to follow these bends but have enough structural support to allow the leads to be pushed and twisted in order to navigate within these veins. Thus, in recent years, there has been a substantial effort to reduce the diameter of endocardial pacing and defibrillation leads. The reduced size of a lead body can facilitate placement of multiple leads through a single blood vessel and also minimize interference between the lead body and the tricuspid valve applicable for leads implanted in the right ventricle, RV.

However, an identified drawback in downsizing the lead body size is the tendency for the lead to rapidly move within the heart chamber in a “whip-like” fashion. This in turn could result in the formation of substantially greater coliform vegetation on the heart wall and the valve leaflets than would be caused by a conventional lead. There is therefore a need to provide a small diameter lead that is stiff enough so as to alleviate the “whipping” action within the chambers.

Furthermore, with increasing complexity of lead systems, there is a growing need for ease of manufacturability of a lead system. A method to easily and inexpensively manufacture a reduced diameter lead body that accommodates access to a desired implant site is warranted and achieved by the present invention.

For the purpose of clarity, the term “guidewire” will be used in this disclosure to denote the components technically known as stylets and guide wires throughout the remainder of this disclosure. Typical of the known prior art is U.S. Pat. No. 6,366,819 to Stokes which discloses a medical lead having a diameter of no greater than 3 French and employing an insulation material that is substantially stiffer than would normally be employed in the context of a permanently implantable cardiac pacing lead is disclosed. A material having a Young's modulus of at least 25,000 pounds per square inch such as Pellethane 2363-75D, or an even stiffer polymer, is said to be used to provide a lead body that does not exhibit a whip-like effect in response to such movement as would occur during a heart beat. In order to prevent the increased stiffness of the lead body from increasing the possibility that the distal lead tip will perforate the heart tissue, the lead is provided with a distal surface that is not reduced in size. This surface reduces the pressure exerted by the distal lead tip upon body tissue to no greater than 3.6 pounds per square inch. The distal tip may carry an electrode of the same or substantially smaller surface area, allowing the lead to retain desirable electrical characteristics for sensing of cardiac depolarizations and delivery of cardiac pacing pulses.

Another disclosure of pertinent prior art is provided in U.S. Publication No. US 2002/0143377 to Wessman et al. In this instance, a lead body and method for lead body manufacture are provided having at least one conductor positioned between an inner insulator and an outer insulator wherein the outer insulator is fused to the inner insulator by heating. Further, an insulating spacer may be provided between the conductors that may be fused to either or both of the outer insulator and the inner insulator.

It was in light of the foregoing that the present invention was conceived and has now been reduced to practice.

### SUMMARY

An implantable cardiac stimulation lead system for use with a stimulation device includes a lead body having an outer diameter of less than 4 French and formed of a biocompatible material having a Young's modulus of at least 20,000 pounds per square inch. In the lead body, an inner insulator is composed of PTFE (polytetrafluoroethylene) with an outer peripheral surface formed with a helical groove containing a conductor extending between a proximal connector and a distal tip electrode, and an outer insulator composed of polyurethane or silicone rubber is positioned around the inner insulator. The lead system may be formed with a lumen for receiving a stylet longitudinally through the lead body. A pair of insulated conductors may be received in the helical groove, one extending to the distal tip electrode, the other extending to a ring electrode proximally spaced from the distal tip electrode.

The assembly of the lead body, according to the present invention, could entail, but not necessarily be restricted to the following operational steps:
1) The conductors, that is, coated or uncoated cables or coils, are wound along the helical grooves running the length of the inner tubing, made of a suitable medical grade implantable material with a high Young's Modulus, therefore, relatively stiff. The configuration of the conductors may be co-radial. The inner tubing may additionally accommodate a lumen for the passage of a stylet or guidewire or inner conductor (in a co-axial design).
2) Additionally, an outer sheath made of a suitable material such as silicone, polyurethane, or a combination of those materials, will be slid over the lead body sub-assembly to hold the conductor or conductors in place.
3) Welding or crimping one set of conductors to establish electrical continuity with the anodal ring electrode; and
4) Flaring the distal end of the other set of conductors and subsequently welding the distal end of the other set of conductors. to the tip electrode.

In another embodiment of the present invention, the flexibility of the lead can be altered over its length by varying the distance between adjacent helical grooves. Additionally, a section of the lead accommodating closely spaced grooves would provide for a more flexible section of the lead and vice versa. This concept lends merit in left heart leads, where approximately 75-80 cm of the proximal section of the lead needs to be relatively stiff to ease maneuverability/torquability/pushability, while the distal 5-10 cm is required to be flexible to allow the distal end of the lead to work its way through the tortuous pathways of the venous system.

In another embodiment, an equivalent inner insulation to PTFE is chosen, that is stiff enough, that is, with a sufficiently high Young's Modulus, to alleviate the “whip-like” action observed in known downsized leads.

A primary feature, then, of the present invention is the provision of a lead assembly connecting implantable medical devices to selected body tissue.

Another feature of the present invention is the provision of such an improved lead assembly in which the lead body size is minimized, preferably less than 4 French.

Yet another feature of the present invention is the provision of such an improved lead assembly having a small diameter lead body and a simplified construction.

Still another feature of the present invention is the provision of such an improved lead assembly that allows the lead to more easily track the veins of the heart.

Still a further feature of an improved lead assembly is the provision of such an improved lead assembly that provides for the use of two leads in an 8 French introducer.

Yet a further feature of the present invention is the provision of such an improved lead assembly which employs inner insulation of PTFE to provide stiffness to the lead and thereby alleviate the “whip-like” action which often occurs with small sized leads implanted in the chambers of the heart.

Yet another feature of the present invention is the provision of such an improved lead assembly in which the lead body has varying flexibility or stiffness along its length.

Other and further features, advantages, and benefits of the invention will become apparent in the following description taken in conjunction with the following drawings. It is to be understood that the foregoing general description and the following detailed description are exemplary and explanatory but are not to be restrictive of the invention. The accompanying drawings which are incorporated in and constitute a part of this invention, illustrate one of the embodiments of the invention, and together with the description, serve to explain the principles of the invention in general terms. Like numerals refer to like parts throughout the disclosure.

### BRIEF DESCRIPTION OF THE DRAWINGS

The foregoing aspects and other features of the present invention are explained in the following description, taken in connection with the accompanying drawings, wherein:

FIG. 1 is a diagrammatic perspective view illustrating an implanted lead system for providing electrical stimulation of a heart employing an implanted lead embodying the present invention;

FIG. 2 is a perspective view, certain portions being cut away and shown in section, of one construction of a known implantable cardiac lead;

FIG. 3 is a perspective view that depicts one of several successive stages of introducing a transvenous endocardial lead into a vein;

FIGS. 4 and 5 are perspective views, with certain parts being shown in section which depict additional successive stages, respectively, of introducing a transvenous endocardial lead into a vein;

FIG. 6 is a perspective view of an implantable lead embodying the invention in combination with a stimulating device such as a pacemaker;

FIG. 7 is a longitudinal elevation view, substantially in cross section, of one portion of a lead embodying the invention;

FIG. 8 is a longitudinal elevation view, substantially in cross section, of another portion of a lead embodying the invention; and

FIG. 9 is a longitudinal elevation view, substantially in cross section and similar to a portion of the lead illustrated in FIG. 8, presenting another embodiment of the invention.

### DETAILED DESCRIPTION OF THE PREFERRED EMBODIMENT

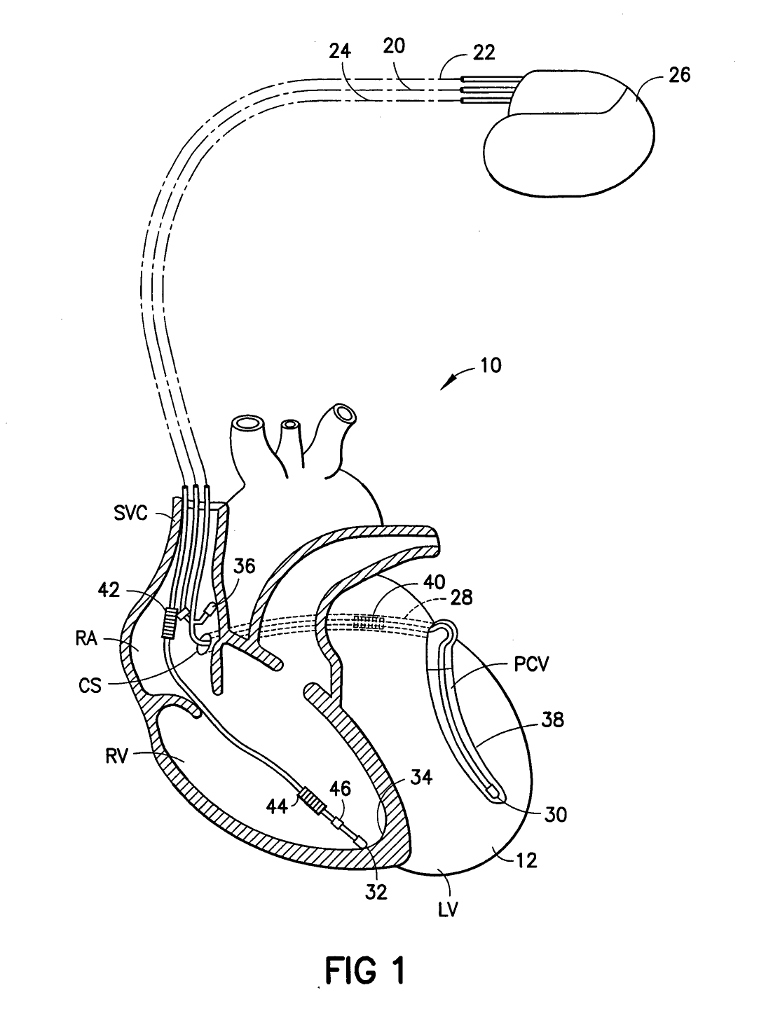

Refer now to the drawings and, initially, to FIG. 1 in which is shown a diagrammatic perspective view of an implanted system 10 for providing electrical stimulation of a heart 12 incorporating features of the present invention. Although the present invention will be described with reference to the embodiments shown in the drawings, it should be understood that the present invention can be embodied in many alternate forms or embodiments. In addition, any suitable size, shape or type of elements or materials could be used.

In FIG. 1, implantable leads 20, 22, 24 are illustrated generally embodying the invention for stimulation of the body, the heart 12 in this instance, by means of a pacemaker 26 or other suitable pulse generating or stimulating device. This is a cross section view of a human heart showing the right atrium RA and the right ventricle RV along with the coronary sinus CS and a vein 28 of the left side of the heart. This vein of the left side could be any of the veins found on the left side of the heart such as the great cardiac vein, posterior vein, or the lateral vein of the left ventricle LV. The leads are shown in a typical placement, lead 20 being an RA lead, lead 22 being an RV lead, and lead 24 being an LV lead inserted via the superior vena cava SVC into the coronary sinus ostium CSO located in the right atrium RA. The lead 24 is then advanced through the coronary sinus ostium, passing through the coronary sinus and placed into a tributary of the coronary venous system, preferably the left posterior cardiac vein PCV with an associated tip electrode 30 being placed deep in the distal portion of the left side of the heart. The phrase “coronary venous system” refers to the coronary sinus vein, great cardiac vein, left marginal vein, left posterior vein, middle cardiac vein, and/or small cardiac veins or any other cardiac vein accessible by the coronary sinus. From this location, the lead 24 can be used to stimulate the left ventricle LV. Clearly, the lead 24 must follow a tortuous path in order for the tip electrode 30 to reach its intended destination. The lead 22 extends to a tip electrode 32 placed in the apex 34 of the right ventricle RV and illustrates the typical position of a lead in the right ventricle. The lead 20 extends to a tip electrode 36 shown in the appendage of the right atrium RA and illustrates the typical position of the lead in the appendage of the right atrium. In this scenario, component 38 is typical of a sensing electrode of the LV lead 24, component 40 is typical of a shock electrode of the LV lead 24, component 42 is typical of a proximal shock coil of the RV lead 22, component 44 is typical of a distal shock coil of the RV lead 22, and component 46 is typical of a ring electrode of the RV lead 22.

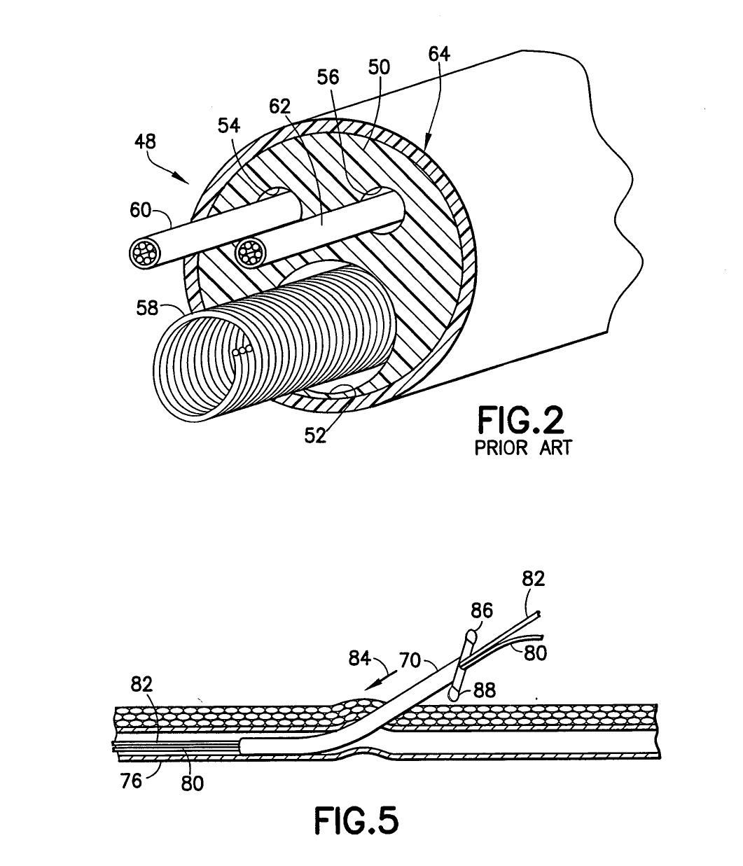

The thrust of the present invention is to provide an implantable lead of a smaller diameter than known leads which will readily negotiate the tortuous paths of the heart or other organ of the body which are necessary in order for an electrode to reach its intended destination. In FIG. 2, one type of known implantable cardiac lead 48 typically utilizes either silicone rubber or polyurethane as the lead's primary insulation material for an insulating sheath 50 formed with longitudinally extending lumina 52, 54, and 56 of circular cross section. A PTFE (polytetrafluoroethylene) coated coil conductor 58 is received in the lumen 52 for connection to a distal electrode such as electrode 32, and redundant ETFE (ethylene-tetrafluoroethylene) coated cable conductors 60 and 62 are received in the lumina 52, 54, respectively, for connection to a ring electrode such as the ring electrode 46. Construction of the lead 48 is completed with the application, optionally, of an outer polyurethane sheath 64 overlying the insulating sheath 50.

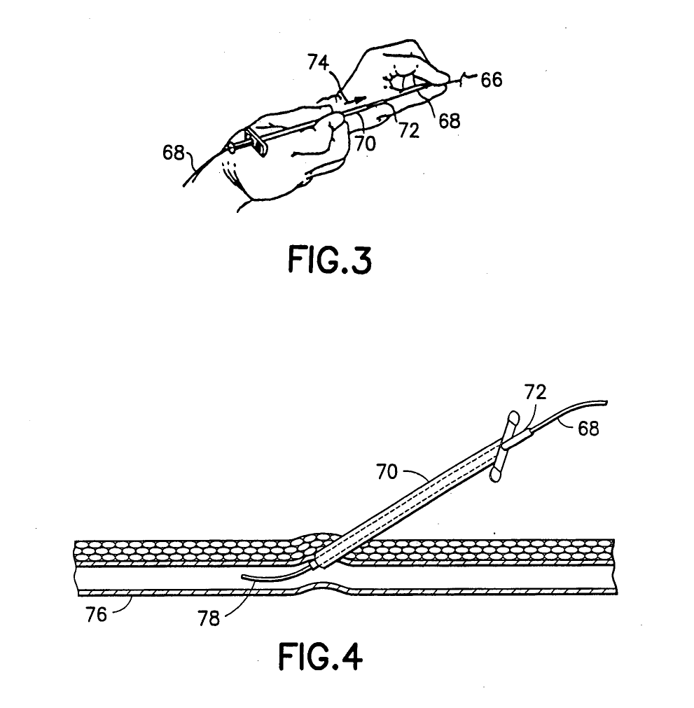

With the aid of FIGS. 3, 4, and 5, a surgical lead implantation procedure will be explained in brief which can be performed for implanting leads embodying the present invention. In the course of this known procedure, a small skin incision 66 (FIG. 3) is made at the entry site of a guide wire 68, parallel to the patient's clavicle (not illustrated). This will become the “pacemaker pocket” at a later stage of the procedure. In the next stage of the implantation procedure, as shown in FIG. 4, an introducer sheath 70 with tapered vessel dilator 72, as an assembly, is threaded onto the proximal end of the guide wire 68. Sheath 70 and dilator 72 are advanced in the direction of an arrow 74, through the subclavian fascia and into subclavian vein 76, until a short length (e.g., 2 to 8-cm) of the introducer sheath 70 and the vessel dilator 72 remain exposed along with the guide wire 68, as shown in FIG. 4.

Next, the vessel dilator 72 is withdrawn and the sheath 70 is introduced further within the subclavian vein 76, leaving the introducer sheath 70 and the guide wire 68 in place with its distal end 78 disposed within the subclavian vein. The guide wire 68 may be removed at this point as well, although it may be left in place in case the lead needs to be repositioned or reinserted or an additional lead needs to be inserted. As shown in FIG. 5, the introducer sheath 70 must bend to conform to the shape of subclavian vein 76 to provide an unobstructed conduit for leads 80, 82 to be introduced. Through such curvature, moreover, leads 80, 82 may be introduced so as to be parallel to the vein 76.

In the final stages of the lead implantation procedure, the pacing leads 80, 82 are inserted into the proximal end of the introducer sheath 70 in the direction of an arrow 84, and advanced into the desired position within the vein 76. Lastly, the introducer sheath 70 is removed which may be accomplished in one of several known ways, depending upon its particular type of construction. In one known manner, the introducer sheath 70 may be longitudinally split by pulling tabs 86 and 88 in opposite directions.

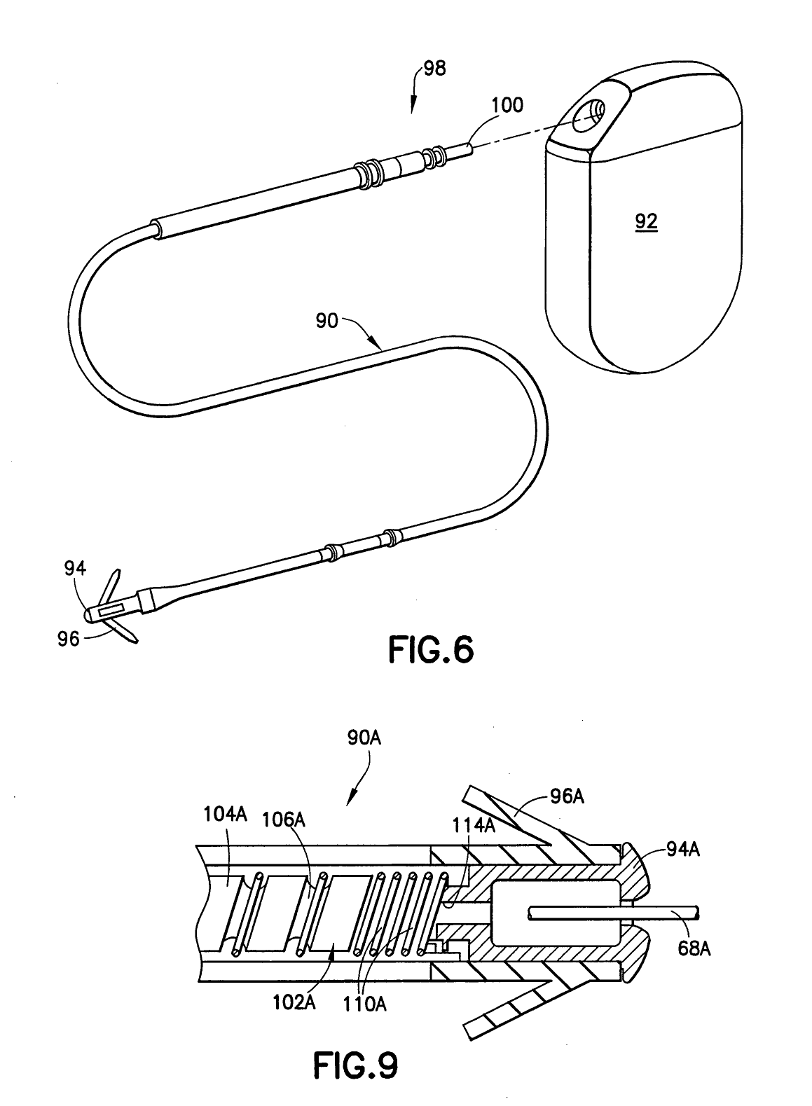

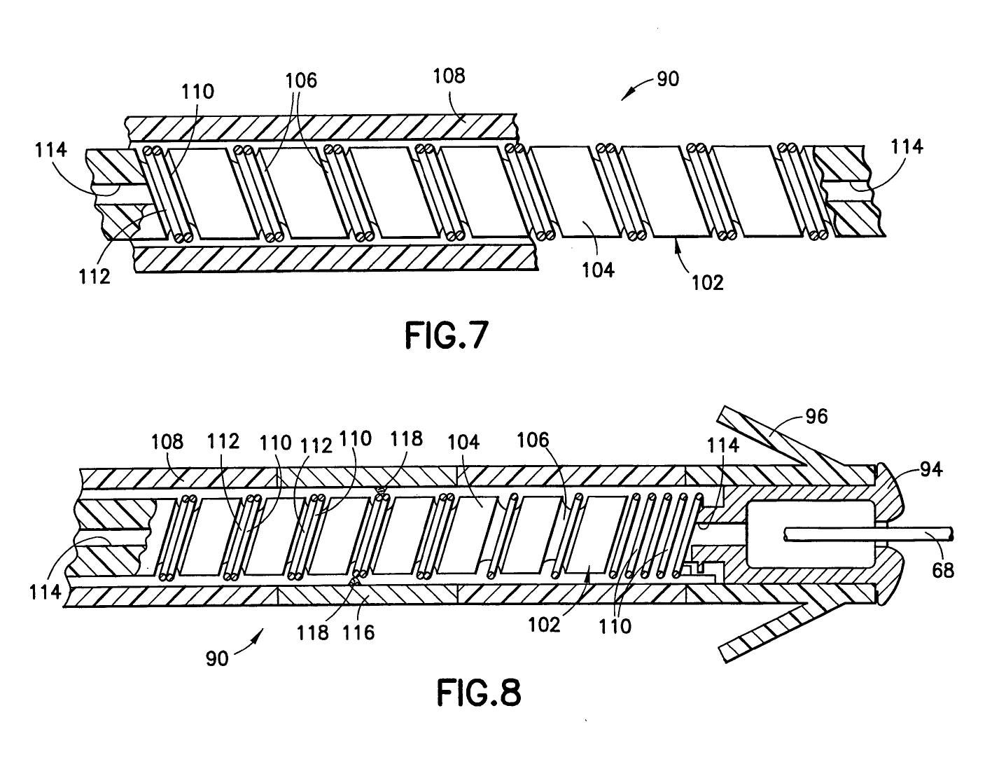

A particular benefit of the present invention is that the improvements provided enable a pair of leads to be introduced simultaneously for implantation using a standard-sized 8 French introducer sheath resulting in a significant reduction of time for the procedure to take place. With this in mind, turn now to FIGS. 6, 7, 8, and 9. FIGS. 7, 8, and 9 illustrate side elevation partially longitudinally-sectioned views at different longitudinal locations of an implantable lead 90 for electrical stimulation of the body and embodying the present invention.

As seen in FIG. 6, the lead 90 is used in combination with an implantable stimulation device 92 such as a pacemaker and interconnects a distal tip electrode 94 to be secured adjacent an interior wall of the heart by tines 96, for example, and an electrical connector 98 at a proximal end 100 for attachment to the stimulation device. The lead 90 includes an inner insulator 102 composed of a suitable polymer, and preferably a fluoropolymer such as PTFE or ETFE and having an outer peripheral surface 104 formed with a helical groove 106. An outer insulator 108 composed typically of polyurethane or silicone rubber or combinations of those materials is positioned around the inner insulator 102 and at least one conductor 110 is received in the helical groove 106 in the region between the inner insulator and the outer insulator. In the instance of a unipolar stimulating system, the lead system would include only one conductor. However, in the instance illustrated, the lead 90 is intended for a bipolar stimulating system such that a second conductor 112 joins the first conductor 110 in the helical groove 106. In this instance, both conductors 110, 112 are provided with a suitable dielectric coating for electrical insulation from one another.

The lead 90 is illustrated as having a lumen 114 for receiving the guide wire 68 longitudinally through the lead. In FIG. 8, the lead 90 is seen to include the distal tip electrode 94 as well as a ring electrode 116 proximally spaced from the distal tip electrode.

The first conductor 110 received in the helical groove 106 connects the proximal connector 98 (FIG. 6) to the distal tip electrode 94. The second conductor 112, also received in the helical groove 106, connects the proximal connector to the ring electrode 116 by a welded joint 118. In one instance, the distal tip electrode 94 is a cathode and the ring electrode 116 is an anode. Oppositely and just as efficaciously for purposes of the invention, in another instance, the distal tip electrode 94 is an anode and the ring electrode 116 is a cathode.

While small diameter pacing leads with highly flexible lead bodies do reduce the possibility of perforation and do facilitate the passage of multiple leads through the same blood vessel, e.g., the cephalic or subclavian vein, it has been determined that very small diameter and very flexible leads tend to cause substantial damage within the heart, due to their tendency to be rapidly moved within the heart chamber in a “whip-like” fashion. This damage may result in the formation of substantially more coliform vegetations (fibrous nodules) on the heart wall and valve leaflets than would be caused by stiffer leads.

The leads of the present invention, however, retain the advantages of a small diameter lead while avoiding damage due to whip-like movement of the lead within the heart chamber. The present invention accomplishes this result by means of a lead employing an insulation material that has a substantially higher Young's modulus (tensile stiffness) than would normally be employed in the context of a permanently implantable cardiac pacing lead.

The properties of the inventive lead structure may be considered using the principal that the flexural stiffness, or bending moment, of a fiber or tube varies with the tensile modulus, and the diameter to the 4^{th} power. That is, for a tube, the bending moment, or flexural stiffness is determined as:

$$M = E \times \frac{{OD}^{4} - {ID}^{4}}{64}$$

wherein M is the bending moment expressed in Newton meters, or equivalent units,
wherein E=Young's modulus, is a measure of tensile stiffness of the material employed to manufacture the tube, and
wherein OD and ID represent the outer and inner diameters, respectively, of the tube.

In this regard, it has been found desirable to form the inner insulation 102 and outer insulation 108 of the lead 90 of a biocompatible material having a Young's modulus of at least 20,000 pounds per square inch.

Continuing with this train of thought, it has been found desirable to tailor the lead 90 to have different stiffness characteristics along its length for a more beneficial result. Thus, for increased stiffness, the pitch of the helical groove 106 in a proximal portion of the lead 90 is relatively fine, the pitch being in the range of about 1 to 10 times the diameter of the conductor for a length in the range of about 1 to 85 cm and, for increased flexibility, the pitch of the helical groove adjacent the distal end of the lead is relatively coarse, the pitch being in the range of about 1 to 15 times the diameter of the conductor for a length in the range of about 1 to 85 cm.

Another embodiment of the invention is illustrated in FIG. 9 as a unipolar version 90A of the lead 90 illustrated in FIG. 8. In this instance, all components of the lead 90A are expressed with reference numerals provided with the suffix “A” to indicate the similar components provided in the bipolar lead 90.

It should be understood that the foregoing description is only illustrative of the invention. Various alternatives and modifications can be devised by those skilled in the art without departing from the invention. Accordingly, the present invention is intended to embrace all such alternatives, modifications and variances which fall within the scope of the appended claims.

1. A lead comprising:
a lead body comprising an inner member defining an outer peripheral surface formed with a helical groove, and an outer insulator positioned around the inner member;
a first electrode and a second electrode mounted on the lead body; and
at least two conductors received in the helical groove between the inner member and the outer insulator, wherein at least one of the at least two conductors is connected to the first electrode, and wherein at least one of the at least two conductors is connected to the second electrode.

2. The lead of claim 1
wherein the inner member is composed of a polymer; and
wherein the outer insulator is composed of a material selected from the group consisting of polyurethane, silicone rubber, and combinations thereof.

3. The lead of claim 2
wherein the polymer is a fluoropolymer.

4. The lead of claim 1 and further comprising:
a proximal connector; and
a distal tip electrode connected to the proximal connector by the conductor.

5. The lead of claim 1, wherein the inner member defines a lumen for receiving a guidewire longitudinally through the lead body.

6. The lead of claim 1 and further comprising:
a proximal connector; and
wherein the first electrode comprises a distal tip electrode; and
wherein the second electrode comprises a ring electrode proximally spaced from the distal tip electrode.

7. The lead of claim 6
wherein the distal tip electrode is a cathode; and
wherein the ring electrode is an anode

8. The lead of claim 1
wherein the first and second conductors have a dielectric coating for electrical insulation from one another.

9. The lead of claim 1
wherein the pitch of the helical groove in a proximal portion of the lead body is relatively fine for increased stiffness thereof; and
wherein the pitch of the helical groove adjacent the distal end of the lead body is relatively coarse for increased flexibility of the distal end of the lead body.

10. An implantable lead system for use with an implantable stimulation device, the lead system comprising:
a lead body comprising an inner member defining an outer peripheral surface formed with a helical groove, and an outer insulator positioned around the inner insulator;
a first electrode and a second electrode mounted on the outer insulator;
at least two conductors received in the helical groove between the inner member and the outer insulator, wherein at least one of the at least two conductors is connected to the first electrode, and wherein at least one of the at least two conductors is connected to the second electrode; and
a proximal connector connected to the lead body and coupled to the first and second electrodes via the respective conductors.

11. The implantable lead system of claim 10 wherein:
the inner member is composed of a polymer; and
the outer insulator is composed of a material selected from the group consisting of polyurethane, silicone rubber, and combinations of polyurethane and silicone rubber.

12. The implantable lead system of claim 10 wherein:
the inner member defines a lumen adapted to receive a guidewire.

13. The implantable lead system of claim 10 wherein the at least two conductors comprise first and second insulated conductors.

14. The implantable lead system of claim 10 wherein the pitch of the helical groove in a proximal portion of the lead body is relatively fine for increased stiffness thereof; and
wherein the pitch of the helical groove adjacent the distal end of the lead body is relatively coarse for increased flexibility of the distal end of the lead body.

15. The implantable lead system of claim 10 wherein the first electrode comprises a distal tip electrode; and
wherein the second electrode comprises a ring electrode proximally spaced from the distal tip electrode.
